(19) United States
(12) Patent Application Publication
(10) Pub. No.: US 2024/0270792 A1
(43) Pub. Date: Aug. 15, 2024

(54) SHORT ANTIMICROBIAL PEPTIDES

(71) Applicants: SORBONNE UNIVERSITE, Paris (FR); CENTRE NATIONAL DE LA RECHERCHE SCIENTIFIQUE, Paris (FR)

(72) Inventors: Ali LADRAM, Ermont (FR); Sonia Maria COSTA ANDRE, Nanterre (FR); Christophe PIESSE, Nanterre (FR); Thierry FOULON, Paris (FR)

(73) Assignees: SORBONNE UNIVERSITE, Paris (FR); CENTRE NATIONAL DE LA RECHERCHE SCIENTIFIQUE, Paris (FR)

(21) Appl. No.: 18/567,846

(22) PCT Filed: Jun. 11, 2021
(86) PCT No.: PCT/IB2021/000411

§ 371 Date: Dec. 7, 2023

(51) Int. Cl. C07K 7/06; A61K 38/00; A61P 31/04; C07K 7/64

(52) U.S. Cl. CPC C07K 7/06; A61P 31/04; C07K 7/64; A61K 38/00

(57) ABSTRACT

The present invention relates to novel short antimicrobial peptides derived from SHf, to pharmaceutical compositions comprising said peptides and to the uses thereof, in particular as medicament, disinfectant, preservative, agent preventing biofilm formation or pesticide.

The present invention relates to novel short antimicrobial peptides, to pharmaceutical compositions comprising said peptides and to the uses thereof, in particular as medicament, disinfectant, preservative, agent preventing biofilm formation or pesticide.

The evolution and spread of antibiotic resistance among bacteria is a major public health problem today, especially in the hospital setting with the emergence of multidrug resistant strains. Intensive research efforts have led to the development of new antibiotics effective against these resistant strains. Nevertheless, through use, mechanisms of resistance to these drugs emerge and limit their efficacy.

In view of this phenomenon, antimicrobial peptides (AMPs) appear very promising for the design of new therapeutic agents. Cationic antimicrobial peptides are thought to be one of the key components of the innate immune system of multicellular organisms, which provides first-line defense against pathogens. The interest of these peptides lies on the one hand in their very broad spectrum of activity enabling in particular their use in the treatment of infections caused by multidrug resistant strains. Secondly, their mode of action is based on permeabilisation or rapid fragmentation of the microorganism membrane and is therefore unlikely to lead to the development of resistance mechanisms.

In particular, AMPs have attracted considerable interest as potential agents against bacterial biofilms. Biofilms are bacteria that stick together, forming a community, which is embedded within a self-produced matrix. Biofilm bacteria show much greater resistance to antibiotics than their free-living counterparts and are responsible for various pathological conditions that are difficult to treat, such as chronic infection of patients affected with cystic fibrosis, endocarditis, cystitis, infections caused by indwelling medical devices and dental plaque formation involved in caries and periodontitis. Since biofilm resistance to antibiotics is mainly due to the slow growth rate and low metabolic activity of bacteria in such community, the use of AMPS appears to be an attractive therapeutic approach because, due to their mode of action, they have a high potential to act also on slow growing or even non-growing bacteria.

Antimicrobial peptides have been identified in plants, insects, amphibia and mammals. Amphibian skin represents a major source of AMPs and every species of frog possesses its specific peptide repertoire generally composed of 10 to 15 AMPs.

Frogs of the Ranidae family are very numerous and this family currently counts 26 genera and 422 species (see https://amphibiansoftheworld.amnh.org/). These frogs synthesize and secrete a remarkable diversity of AMPs, which have been classified into 12 families (Conlon, “Structural diversity and species distribution of host-defense peptides in frog skin secretions”, Cell. Mol. Life Sci., 2011 July, 68:2303-15; Ladram and Nicolas, “Antimicrobial peptides from frog skin: biodiversity and therapeutic promises”, Front. Biosci. (Landmark Ed), 2016, 21:1341-71). One such family, the temporins, comprises AMPs of small size (generally 13-14 residues) the sequences of which vary widely according to species. More than 100 members of the temporin family have been identified. These temporins have been isolated from several Rana species such as for example Rana temporaria (Simmaco et al., “Temporins, antimicrobial peptides from the European red frog Rana temporaria”, Eur. J. Biochem., 1996, 242: 788-92), Rana esculents (Simmaco et al., “Purification and characterization of bioactive peptides from skin extract of Rana esculenta”, Biochem. Biophys. Acta, 1990, 1033: 318-23), Rana japonica (Isaacson et al., “Antimicrobial peptides with atypical structural features from the skin of the Japanese brown frog Rana japonica”, Peptides, 2002, 23: 419-25), Rana ornativentris (Kim et al., “Antimicrobial peptides from the skin of the Japanese mountain brown frog, Rana ornativentris”, J. Pept. Res., 2001, 58: 349-56) and Pelophylax (Rana) saharicus (Abbassi et al., “Isolation, characterization and molecular cloning of new temporins from the skin of the North African ranid Pelophylax saharica”, Peptides, 2008, 29: 1526-33; Abbassi et al., “Temporin-SHf, a new type of phe-rich and hydrophobic ultrashort antimicrobial peptide”, J. Biol. Chem., 2010, 285: 16880-92; Abbassi et al., “Antibacterial and leishmanicidal activities of temporin-SHd, a 17-residue long membrane-damaging peptide”, Biochimie, 2013, 95: 388-9).

Unlike the other 12 families of Ranidae peptides, the temporins lack the “Rana box” motif, a C-terminal heptapeptide domain cyclized by a disulphide bridge (Mangoni, “Temporins, anti-infective peptides with expanding properties”, Cell. Mol. Life Sci., 2006, 63: 1060-9). Furthermore, the majority of temporins contain a single basic residue, which confers a net charge of +2 at physiological pH. Generally, the temporins are particularly active against Gram-positive bacteria and yeasts but they also exhibit antifungal properties (Rollins-Smith et al., “Activities of temporin family peptides against the chytrid fungus (Batrachochytrium dendrobatidis) associated with global amphibian declines”, Antimicrob. Agents Chemother., 2003, 47: 1157-60) and, for some, antiviral properties (Chinchar et al., “Inactivation of viruses infecting ectothermic animals by amphibian and piscine antimicrobial peptides”, Virology, 2004, 323: 268-75; Marcocci et al., “The amphibian antimicrobial peptide temporin B inhibits in vitro Herpes Simplex Virus 1 infection”, Antimicrob. Agents Chemother., 2018, 62:e02367-17; Roy et al., “Comparison of anti-viral activity of frog skin anti-microbial peptides temporin-Sha and [K³]SHa to LL-37 and temporin-Tb against Herpes Simplex Virus type 1”, Viruses, 2019, 11:77).

It was found that temporin-SHa isolated from the skin of the North African frog Pelophylax saharicus exhibits antiparasitic activity against protozoa belonging to the genus Leishmania, which are the causal agents of leishmaniasis (Abbassi et al., “Isolation, characterization and molecular cloning of new temporins from the skin of the North African ranid Pelophylax saharica”, Peptides, 2008, 29: 1526-33). Based on this finding, analogs of said temporin exhibiting improved antimicrobial activity were obtained by substitution of one or more amino acids of the polar face of the α-helix by a basic amino acid (WO2010/106293 and WO2015/044356). The Authors demonstrated that analogs of temporin-SHa have a reduced cytotoxicity.

Temporin-SHf (SHf) is an atypical AMP also isolated from the frog Pelophylax saharicus, which has the characteristics of being the smallest natural temporin and a phenylalanine-rich peptide (Abbassi et al., “Temporin-SHf, a new type of phe-rich and hydrophobic ultrashort antimicrobial peptide”, J. Biol. Chem., 2010, 285: 16880-92; André et al., “Structure-activity relationship-based optimization of small temporin-SHf analogs with potent antibacterial activity”, ACS Chem. Biol., 2015, 10: 2257-66) have synthetized SHf analogs, showing for some analogs an antimicrobial activity against Gram-positive and/or Gram-negative bacteria with a non-hemolytic activity. The Authors demonstrated that the analog [p-^{t}BuF², R⁵]SHf is non-cytotoxic and has an antimicrobial activity against Gram-positive and Gram-negative bacteria, except for Klebsiella pneumoniae. They also demonstrated that a α-MeF³ analog of SHf has a strong activity against Gram-positive and Gram-negative bacteria, including Klebsiella pneumoniae, but exhibits a higher hemolytic activity. None of these SHf analogs showed both a strong antimicrobial activity against the clinically relevant strains and a reduced cytotoxicity.

Therefore, there is still a great need for improved AMPS exhibiting strong antimicrobial activity with greatly reduced toxicity against mammalian cells. There is also a need to reduce the size of these AMPs to produce them more easily and low costly.

Interestingly, the Inventors have designed new short SHf analogs with an extended antimicrobial activity against Gram-positive and Gram-negative bacteria combined to a reduced hemolytic activity.

The invention aims to provide novel antimicrobial peptides, analogs of temporin-SHf.

Accordingly, the present invention relates to a peptide exhibiting an antimicrobial activity of sequence X1-X2-X3-X4-X5-X6-X7-X8 (SEQ ID No 1), wherein:
X1 is an amino acid selected from the group consisting of F (phenylalanine), hF (homophenylalanine), (C₁-C₄ alkyl)F (C₁-C₄ alkyl phenylalanine) and preferably p-^{t}BuF (4-tert-butyl-phenylalanine), 4a-F (4-amino phenylalanine), W (tryptophan), R (arginine) and K (lysine),
X2 is an amino acid selected from the group consisting of F (phenylalanine), I (isoleucine), W (tryptophan), hF (homophenylalanine), K (lysine), (C₁-C₄ alkyl)F (C₁-C₄ alkyl phenylalanine) and preferably p-^{t}BuF (4-tert-butyl-phenylalanine), L (leucine) and R (arginine),
X3 is an amino acid selected from the group consisting of F (phenylalanine), K (lysine), hF (homophenylalanine), R (arginine), (C₁-C₄alkyl)F (C₁-C₄ alkyl phenylalanine) and preferably p-^{t}BuF (4-tert-butyl-phenylalanine) and W (tryptophan),
X4 is an amino acid selected from the group consisting of L (leucine), F (phenylalanine), R (arginine), hF (homophenylalanine) and W (tryptophan),
X5 is an amino acid selected from the group consisting of S (serine), HmS (α-hydroxymethyl serine), R (arginine), K (lysine) and hF (homophenylalanine),
X6 is an amino acid selected from the group consisting of R (arginine) and K (lysine),
X7 is an amino acid selected from the group consisting of I (isoleucine), F (phenylalanine), R (arginine), W (tryptophan) and hF (homophenylalanine),
X8 is an amino acid selected from the group consisting of F—NH₂ (phenylalanine amide) and R—NH₂ (arginine amide);
   and pharmaceutically acceptable salts of said peptide,
   and wherein the following peptides are excluded:
FFFLSRIFamide (SEQ ID No 2),
FFFLRRIFamide (SEQ ID No 3),
FFFLRRIFacid (SEQ ID No 4),
FSFLSRIFamide (SEQ ID No 5),
FFFL(HmS)RIFamide (SEQ ID No 6),
FF(α-MeF)LSRIFamide (SEQ ID No 7),
(p-^{t}BuF)FFLSRIFamide (SEQ ID No 8),
(p-^{t}BuF)FFLRRIFamide (SEQ ID No 9),
F(p-^{t}BuF)FLSRIFamide (SEQ ID No 10),
F(p-^{t}BuF)FLRRIFamide (SEQ ID No 11),
FF(p-^{t}BuF)LSRIFamide (SEQ ID No 12),
FF(p-^{t}BuF)LRRIFamide (SEQ ID No 13).

HmS is a α-hydroxymethyl serine. This amino acid has the following structure:

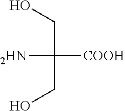

α-MeF is a α-methyl-phenylalanine. This amino acid has the following structure:

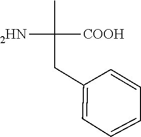

The amino acid hF (homophenylalanine) brings an additional —CH₂— on its lateral chain compared to F. This residue is more hydrophobic than the amino acid F. This amino acid has the following structure:

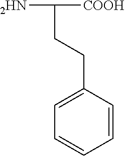

The amino acid p-^{t}BuF 4-tert-butyl-phenylalanine) has the following structure:

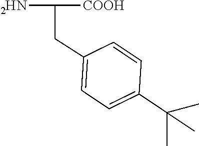

The amino acid 4a-F (4-amino-phenylalanine) has the following structure:

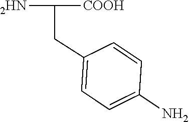

The term “microbe” or “microbial” as employed herein refers to bacteria, fungi, yeasts, viruses and/or parasites.

The term “microbial infection” as employed herein refers to an infection caused by bacteria, fungi, yeasts, viruses and/or parasites.

The term “antimicrobial activity” as employed herein refers to an antibacterial, antiviral, antifungal and/or antiparasitic activity. Said activity may be evaluated by measuring different parameters such as IC50 or MIC.

“IC50” or “half maximal inhibitory concentration” is the concentration of a substance needed to reduce the growth in vitro of a population of microorganisms by half. “MIC” or “minimum inhibitory concentration” is the lowest concentration of a substance that will totally inhibit microbial growth after 18-24 hours of incubation, generally at 37° C., in the presence of said substance.

The invention also encompasses the pharmaceutically acceptable salts of a peptide according to the invention. Pharmaceutically acceptable salts may, for example, be salts of pharmaceutically acceptable mineral acids such as hydrochloric acid, hydrobromic acid, sulphuric acid and phosphoric acid; salts of pharmaceutically acceptable organic acids such as acetic acid, citric acid, maleic acid, malic acid, succinic acid, ascorbic acid and tartaric acid; salts of pharmaceutically acceptable mineral bases such as salts of sodium, potassium, calcium, magnesium or ammonium; or salts of organic bases which contain a salifiable nitrogen, commonly used in pharmaceutical technique. The methods for preparing said salts are well known to one of skill in the art.

According to a preferred embodiment, the antimicrobial peptide of sequence X1-X2-X3-X4-X5-X6-X7-X8 (SEQ ID No 1), wherein:
X1 is an amino acid selected from the group consisting of F, hF, p-^{t}BuF, 4a-F, W, R and K,
X2 is an amino acid selected from the group consisting of F, I, W, hF, K, p-^{t}BuF, L and R,
X3 is an amino acid selected from the group consisting of F, K, hF, R, p-^{t}BuF and W,
X4 is an amino acid selected from the group consisting of L, F, R, hF and W,
X5 is an amino acid selected from the group consisting of S, HmS, R, K and hF,
X6 is an amino acid selected from the group consisting of R and K,
X7 is an amino acid selected from the group consisting of I, F, R, W and hF,
X8 is an amino acid selected from the group consisting of F_{amide} and R_{amide},

and wherein the peptides of SEQ ID No 2 to 13 are excluded.

According to a preferred embodiment, the antimicrobial peptide of the invention comprises the sequence X1-X2-X3-X4-X5-X6-X7-X8 (SEQ ID No 1), wherein
X1 is the amino acid F,
X2 is the amino acid p-^{t}BuF,
X3 is an amino acid selected from the group consisting of K, hF and R,
X4 is an amino acid selected from the group consisting of L and F,
X5 is the amino acid R,
X6 is an amino acid selected from the group consisting of R and K,
X7 is an amino acid selected from the group consisting of I and F,
X8 is the amino acid F_{amide},

and wherein the peptide of SEQ ID No 11 is excluded.

According to this embodiment, preferred AMPS are SEQ ID No 25, 29 and 30 and preferably SEQ ID No 25.

According to a preferred embodiment, the AMP has a positive net charge at pH 7 and preferably the positive net charge is at least +2, and more preferably +3, +4 or +5. The positive net charge is calculated within the method provided by the peptide property calculator (https://pepcalc.com/).

In a preferred embodiment, the value of the hydrophobicity of the AMP is comprised from 50 to 80%, preferably 60 to 80%, more preferably 70 to 80% and even more preferably 75%. The value of hydrophobicity may be calculated within the method provided by the peptide hydrophobicity/hydrophilicity analysis (see https://www.peptide2.com/N_peptide_hydrophobicity_hydrophilicity.php).

According to a preferred embodiment, the AMP comprises a sequence with at least three F (phenylalanine).

According to a particular embodiment, the present invention relates to an AMP as defined above in a cyclic form in which the first amino acid X1 is covalently linked to the last X8 amino acid, the peptide SEQ ID No 22 is a cyclic peptide.

In a preferred embodiment, the AMP comprises a sequence with:
3 amino acids F (phenylalanine) and 2 amino acids R (arginine) or 3 amino acids K (lysine), or
4 amino acids F (phenylalanine) and 1 or 2 or 4 amino acids R (arginine), or
5 amino acids F (phenylalanine) and 2 or 3 amino acids R (arginine) or 3 amino acids K (lysine), or
6 amino acids F (phenylalanine) and 1 or 2 amino acids R (arginine).

According to a preferred embodiment, at least one amino acid F (phenylalanine) is substituted by a homophenylalanine (hF) and/or by a (C₁-C₄ alkyl)F. Preferably, the (C₁-C₄ alkyl)F is a 4-tert-butyl-phenylalanine (p-^{t}BuF).

The term “substitution”, as used herein in relation to a position or amino acid, means that the amino acid in the particular position has been replaced by another amino acid or that an amino add different from the one of SEQ ID No 1 is present.

The amino acids constituting the AMP of the invention may be in the L or D configuration, preferably the L configuration.

According to a preferred embodiment, the AMP comprises a sequence selected from the group consisting of SEQ No 14 to SEQ ID No 79 (see Table 1).

TABLE 1
List of peptides

| | SEQ ID | % | Net charge |
|---|---|---|---|
| Peptide Sequence | No | Hydrophobicity | (pH 7) |
| FFFLKKIF_{amide} | 14 | 75 | +3 |
| FIFLRRIF_{amide} | 15 | 75 | +3 |
| FWFLRRIF_{amide} | 16 | 75 | +3 |
| F(hF)KLRRIF_{amide} | 17 | 62.5 | +4 |
| FF(hF)LSRIF_{amide} | 18 | 75 | +2 |
| FF(hF)LRRIF_{amide} | 19 | 75 | +3 |
| F(hF)FLRRIF_{amide} | 20 | 75 | +3 |
| FF(hF)LRKIF_{amide} | 21 | 75 | +3 |
| 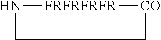 | 22 | 50 | +4 |
| FK(hF)LKKIF_{amide} | 23 | 62.5 | +4 |
| F(p-^{t}BuF)(hF)LRRIF_{amide} | 24 | 75 | +3 |
| F(p-^{t}BuF)(hF)LRKIF_{amide} | 25 | 75 | +3 |
| F(p-^{t}BuF)(hF)LKKIF_{amide} | 26 | 75 | +3 |
| F(p-^{t}BuF)FFRRFF_{amide} | 27 | 75 | +3 |
| F(p-^{t}BuF)FFKRFF_{amide} | 28 | 75 | +3 |
| F(p-^{t}BuF)RFRRFF_{amide} | 29 | 62.5 | +4 |
| F(p-^{t}BuF)KFRRFF_{amide} | 30 | 62.5 | +4 |
| F(p-^{t}BuF)FFKKFF_{amide} | 31 | 75 | +3 |
| F(p-^{t}BuF)KFKKFF_{amide} | 32 | 62.5 | +4 |
| FK(p-^{t}BuF)LKKIF_{amide} | 33 | 62.5 | +4 |
| FFFFSRFF_{amide} | 34 | 75 | +2 |
| FFRFSRFF_{amide} | 35 | 62.5 | +3 |
| FFKFSRFF_{amide} | 36 | 62.5 | +3 |
| F(hF)FFSRFF_{amide} | 37 | 75 | +2 |
| FFFRRRFF_{amide} | 38 | 62.5 | +4 |
| FFFFRRFF_{amide} | 39 | 75 | +3 |
| FFFFrrFF_{amide} | 40 | 75 | +3 |
| FFRRFFFF_{amide} | 41 | 75 | +3 |
| FFFFKRFF_{amide} | 42 | 75 | +3 |
| FFFFRKFF_{amide} | 43 | 75 | +3 |
| FFFFKKFF_{amide} | 44 | 75 | +3 |
| FFFFkkFF_{amide} | 45 | 75 | +3 |
| FWFFRRFF_{amide} | 46 | 75 | +3 |
| FFWFRRFF_{amide} | 47 | 75 | +3 |
| FLFFRRFF_{amide} | 48 | 75 | +3 |
| FRFFRRFF_{amide} | 49 | 62.5 | +4 |
| FFRFRRFF_{amide} | 50 | 62.5 | +4 |
| F(hF)FRRFF_{amide} | 51 | 75 | +3 |
| F(hF) KFRRFF_{amide} | 52 | 62.5 | +4 |
| FF(hF)FRRFF_{amide} | 53 | 75 | +3 |
| FF(hF)FKRFF_{amide} | 54 | 75 | +3 |
| FRFRFRFF_{amide} | 55 | 62.5 | +4 |
| FRFRFRFR_{amide} | 56 | 50 | +5 |
| FRF(hF)RR(hF) F_{amide} | 57 | 62.5 | +4 |
| FRFR(hF)R(hF) R_{amide} | 58 | 50 | +5 |
| FR(hF)FRRFF_{amide} | 59 | 62.5 | +4 |
| FF(hF)FKKFF_{amide} | 60 | 75 | +3 |
| FF(hF)LKKIF_{amide} | 61 | 75 | +3 |
| FFF(hF)RR(hF) F_{amide} | 62 | 75 | +3 |
| FF(hF)LRRRF_{amide} | 63 | 62.5 | +4 |
| FFWLRRRF_{amide} | 64 | 62.5 | +3 |
| (p-^{t}BuF)FKLRRIF_{amide} | 65 | 62.5 | +4 |
| (p-^{t}BuF)KFLRRIF_{amide} | 66 | 62.5 | +4 |
| 4a-FFFFSRFF_{amide} | 67 | 75 | +2 |
| 4a-F(hF)FFSRFF_{amide} | 68 | 75 | +2 |
| 4a-FLFFSRFF_{amide} | 69 | 75 | +2 |
| 4a-FLFFRRFF_{amide} | 70 | 75 | +3 |
| WFFFRRFF_{amide} | 71 | 75 | +3 |
| RFFFRRFF_{amide} | 72 | 62.5 | +4 |
| RFF(hF)RR(hF) F_{amide} | 73 | 62.5 | +4 |
| RFFWRRWF_{amide} | 74 | 62.5 | +4 |
| RF(hF)FRRFF_{amide} | 75 | 62.5 | +4 |
| KF(hF)LKKIF_{amide} | 76 | 62.5 | +4 |
| (hF)R(hF)R(hF)R(hF) R_{amide} | 77 | 50 | +5 |
| (hF)FFLRRIF_{amide} | 78 | 75 | +3 |
| (hF)FKLRRIF_{amide} | 79 | 62.5 | +4 |

Preferably, AMPs of the invention are chosen among peptides of SEQ ID No 19, 25, 29, 30, 32, 33, 34, 38, 39, 41, 42, 43, 49, 50, 51, 53, 55, 56 65, 66 or 72 and more preferably AMPs of the invention have the SEQ ID No 55 or 56.

According to another particular embodiment, AMPs according to the invention have a sequence of SEQ ID No 1 as defined above wherein X2 is not a p-^{t}BuF or wherein X5 is not an arginine (R), wherein peptides of SEQ ID No 3, 4, 9, 10, 11 and 13 are excluded. According to this embodiment, AMPs are chosen among the SEQ ID No 19, 32, 33, 34, 38, 39, 41, 42, 43, 49, 50, 51, 53, 55, 56, 65, 66 or 72. According to this particular embodiment, AMPs according to the invention have preferably a sequence of SEQ ID No 1 wherein X2 is not a p-^{t}BuF and wherein X5 is not an arginine (R). According to this embodiment, AMPS are chosen among the SEQ ID No 33, 34, 41, 42, 55 or 56.

The AMP according to the invention may be obtained by classical chemical synthesis (in solid phase or homogeneous liquid phase, see Behrendt et al., “Advances in Fmoc solid-phase peptide synthesis”, J Pept Sci., 2016, 22:4-27) or by enzymatic synthesis (Bongers and Heimer, “Recent applications of enzymatic peptide synthesis”, Peptides, 1994, 15:183-93).

It may also be obtained by the method consisting in culturing a host cell, such as described hereinafter, comprising a transgene coding for the peptide and expressing said peptide, and extracting said peptide from said host cells or from the culture medium into which the peptide was secreted. With this method, only AMPs with natural amino acids can be obtained.

In another aspect, the present invention relates to a nucleic acid coding for the AMP according to the invention, an expression cassette or an expression vector comprising said nucleic acid. The present invention further relates to a host cell comprising said nucleic acid, expression cassette or expression vector.

“Nucleic acid” is understood to mean any molecule based on DNA or RNA. These may be synthetic or semi-synthetic, recombinant molecules, possibly amplified or cloned into vectors, chemically modified, comprising non-natural bases or modified nucleotides comprising for example a modified bond, a modified purine or pyrimidine base, or a modified sugar.

The nucleic acid according to the invention may be in the form of DNA and/or RNA, single stranded or double stranded. According to a preferred embodiment, the nucleic acid is an isolated DNA molecule, synthesized by recombinant techniques well known to one of skill in the art.

The nucleic acid according to the invention may be deduced from the sequence of the peptide according to the invention and codon usage may be adapted according to the host cell in which the nucleic acid shall be transcribed. These steps may be carried out according to methods well known to one of skill in the art and some of which are described in the reference manual “Molecular cloning: a laboratory manual” (Sambrook et al., Third Edition Cold Spring Harbor, 2001).

The present invention further relates to an expression cassette comprising a nucleic acid according to the invention operably linked to the sequences required for its expression. In particular, the nucleic acid may be under the control of a promoter allowing its expression in a host cell. Generally, an expression cassette is constituted of or comprises a promoter allowing initiation of transcription, a nucleic acid according to the invention, and a transcription terminator. The term “expression cassette” denotes a nucleic acid construct comprising a coding region and a regulatory region, operably linked. The expression “operably linked” indicates that the elements are combined in such a way that the expression of the coding sequence (the gene of interest) and/or the targeting of the encoded peptide are under the control of the transcriptional promoter and/or signal peptide. Typically, the promoter sequence is placed upstream of the gene of interest, at a distance therefrom, which is compatible with the control of expression. Likewise, the sequence of the signal peptide is generally fused upstream of the sequence of the gene of interest, and in the same reading frame with the latter, and downstream of any promoter. Spacer sequences may be present, between the regulatory elements and the gene, as long as they do not prevent expression and/or targeting. In a preferred embodiment, said expression cassette comprises at least one “enhancer” activating sequence operably linked to the promoter.

The present invention also relates to an expression vector comprising a nucleic acid or an expression cassette according to the invention. Said expression vector may be used to transform a host cell and enables the expression of the nucleic acid of the invention in said cell.

The vector may be a DNA or an RNA, circular or not, single- or double-stranded. Advantageously it is selected from among a plasmid, a phage, a phagemid, a virus, a cosmid and an artificial chromosome.

Advantageously, the expression vector comprises regulatory elements allowing the expression of the nucleic acid according to the invention. These elements may contain for example transcriptional promoters, transcriptional activators, terminator sequences, initiation and termination codons. The methods for selecting said elements according to the host cell in which expression is desired, are well known to one of skill in the art.

The vector may also contain elements enabling its selection in the host cell such as, for example, an antibiotic resistance gene or a selectable gene providing complementation of the respective gene deleted from the host cell genome. Such elements are well known to one of skill in the art and extensively described in the literature.

When the host cell to be transformed is a plant cell, the expression vector is preferably a plant vector. Examples of plant vectors are described in the literature, including in particular the T-DNA plasmids of A. tumefaciens pBIN19 (Bevan, “Binary Agrobacterium vectors for plant transformation”, Nucleic Acids Res., 1984, 12: 8711-21), pPZPIOO (Hajdukewicz et al., “The small, versatile pPZP family of Agrobacterium binary vectors for plant transformation”, Plant Mol. Biol., 1994, 25: 989-94), the pCAMBIA series (R. Jefferson, CAMBIA. Australia). The vectors of the invention may additionally comprise an origin of replication and/or a selectable marker gene and/or a plant recombination sequence.

The vectors may be constructed by the classical techniques of molecular biology, well known to one of skill in the art.

The present invention relates to the use of a nucleic acid, an expression cassette or an expression vector according to the invention to transform or transfect a cell. The host cell may be transformed/transfected in a transient or stable manner and the nucleic acid, cassette or vector may be contained in the cell in the form of an episome or in chromosomal form. The present invention relates to a host cell comprising a nucleic acid, a cassette or an expression vector according to the invention.

According to one embodiment, the host cell is a microorganism, preferably a bacterium or a yeast. According to another embodiment, the host cell is an animal cell, for example a mammalian cell such as COS or CHO cells (U.S. Pat. Nos. 4,889,803; 5,047,335). In a particular embodiment, the cell is non-human and non-embryonic.

According to yet another embodiment, the host cell is a plant cell. The term “plant cell” as employed herein refers to any cell coming from a plant and which may constitute undifferentiated tissues such as calluses, and differentiated tissues such as embryos, plant parts, plants or seeds.

The present invention also relates to a method for producing an antimicrobial peptide according to the invention comprising transforming or transfecting a cell with a nucleic acid, an expression cassette or an expression vector according to the invention; culturing the transfected/transformed cell; and recovering the peptide produced by said cell. Methods for producing recombinant peptides are well known to one of skill in the art. For example, one may cite the specific methods described in WO 01/70968 for a production in an immortalized human cell line, WO 2005/123928 for a production in a plant and US 2005-229261 for a production in the milk of a transgenic animal. The present invention also relates to a method for producing an antimicrobial peptide according to the invention comprising inserting a nucleic acid, a cassette or an expression vector according to the invention in an in vitro expression system also called acellular and recovering the peptide produced by said system. Many in vitro or acellular expression systems are commercially available and the use of said systems is well known to one of skill in the art.

The present invention also relates to an antibody specifically binding to a peptide according to the invention.

The present invention relates to an antibody specific of the peptide according to the invention. The term “antibody” as employed herein refers in particular to polyclonal or monoclonal antibodies, fragments thereof (for example the fragments F (ab)′2, F(ab)), single chain antibodies or minibody or else any polypeptide comprising a domain of the initial antibody recognizing the peptide of the invention, particularly CDRs (complementarity determining regions). For example, these are chimeric, humanized or human antibodies. Monoclonal antibodies may be prepared from hybridomas according to methods well known to one of skill in the art. The different methods for preparing antibodies are well known to one of skill in the art.

The present invention also relates to the use of an antibody according to the invention for detecting a peptide according to the invention. It further relates to the use of an antibody according to the invention for making quantitative measurements of a peptide according to the invention, in particular for immunological assays. Said measurements can allow in particular a determination of the expression of the peptide of the invention in a host cell or a transgenic plant according to the invention.

In a further aspect, the present invention relates to a pharmaceutical composition comprising at least one AMP according to the invention, and a pharmaceutically acceptable support and/or excipient.

Said pharmaceutical composition may comprise a mixture of at least two or more AMPs of the invention, preferably it comprises a mixture of at least two AMPs selected in the group consisting of SEQ ID No 25, 29 and 30.

The pharmaceutically acceptable support can be fabrics, non-woven fabrics or medical devices, which are in direct contact with the skin or mucosae. The peptide of the invention can be incorporated into them. These supports release the peptides of the invention either by biodegradation of the anchorage system to the fabric, non-woven fabric or medical devices or by the friction of the latter with the body, by body moisture, by pH of the skin or by body temperature. Likewise, the fabrics and non-woven fabrics can be used to make garments which are in direct contact with the body. Preferably, the fabrics, non-woven fabrics and medical devices containing the peptides of the invention are used for the treatment and/or care of those conditions, disorders and/or pathologies of the skin or mucosae.

Preferred fabrics, non-woven fabrics, garments, medical devices are bandages, gauzes, T-shirts, socks, pantyhose, underwear, girdles, gloves, diapers, sanitary napkins, dressings, wound dressing, bedcovers, wipes, hydrogels, adhesive patches, non-adhesive patches, microelectric patches and/or face masks.

The pharmaceutically acceptable excipients that can be used in the composition according to the invention are well known to one of skill in the art (Gennaro, “Remington's Pharmaceutical Sciences, 18^{th} edition”, Mack Publishing Company, 1990; Frokjaer and Hovgaard, “Pharmaceutical Formulation Development of Peptides and Proteins”, Taylor & Francis, 2000; Kibbe, “Handbook of Pharmaceutical Excipients, 3^{rd} edition”, A Pharmaceutical Press, 2000) and comprise in particular physiological saline solutions and phosphate buffers.

The pharmaceutical composition according to the invention may be suitable for local or systemic administration, in particular for oral, sublingual, cutaneous, subcutaneous, intramuscular, intravenous, intraperitoneal, topical, intra-tracheal, intranasal, transdermal, rectal, intraocular or intra-auricular administration. Preferably, the pharmaceutical composition according to the invention is suitable for cutaneous, oral, topical, or transdermal administration.

According to a particular embodiment, the pharmaceutical composition according to the invention is suitable for topical administration.

The pharmaceutical composition according to the invention may be in the form of tablets, capsules, soft capsules, granulates, suspensions, emulsions, solutions, gels, pastes, ointments, creams, plasters, potions, suppositories, enemas, injectables, implants, patches, sprays or aerosols.

According to one embodiment, the composition according to the invention comprises from 1 to 2000 mg of peptide according to the invention. Preferably, the composition according to the invention comprises from 10 to 100, 150, 200, 250, 500, 750, 1000 or 1500 mg of peptide according to the invention.

The composition according to the invention may further comprise additional active substances, such as other antimicrobial agents, in particular AMPS or antibiotics. The composition may also additionally comprise substances that can potentiate the activity of the peptide according to the invention.

The present invention further relates to the AMP according to the invention, as medicament. Preferably, the medicament is intended for treating an infection caused by a bacterium, a virus, a fungus or a parasite.

The microbial infection may be due to Gram-negative bacteria. In particular, Gram-negative bacteria may be selected from the group consisting of Escherichia coli and bacteria from the genus Pseudomonas, Salmonella, Acinetobacter or Klebsiella. Preferably, Gram-negative bacteria are selected from the group consisting of Escherichia coli, Pseudomonas aeruginosa, Salmonella enterica, Acinetobacter baumannii and Klebsiella pneumoniae.

The microbial infection may be due to Gram-positive bacteria. In particular, Gram-positive bacteria may be selected from the group consisting of bacteria from the genus Staphylococcus, Streptococcus, Listeria or Enterococcus. Preferably, Gram-positive bacteria are selected from the group consisting of Staphylococcus aureus, Streptococcus pyogenes, Listeria ivanovii and Enterococcus faecalis.

The microbial infection may also be due to a fungus. In particular, the fungus may be from the genus Candida or Aspergillus. For example, the fungus may be selected from the group consisting of Candida albicans and Candida parapsilosis.

According to a particular embodiment, the treatment may be curative or preventive.

The subject to be treated is an animal, preferably a mammal. According to a particular embodiment, the subject to be treated is a human. According to another embodiment, the subject to be treated is a domestic animal, breeding animals, livestock or working animals. This veterinary use is to treat microbial infections avoiding antibiotics. Biofilms are responsible for approximately 60% of nosocomial infections. They are essentially due to microbial colonisation of implanted biomaterials. Eradication of a bacterial biofilm is a major clinical problem considering that antibiotics normally active on bacteria in planktonic state often turn out to be much less effective against structures organized into a biofilm. The effect of AMPS on this type of biofilm has been demonstrated in previous studies carried out with temporin-A (Cirioni et al., “Prophylactic efficacy of topical temporin A and RNAIII inhibiting peptide in a subcutaneous rat Pouch model of graft infection attributable to Staphylococci with intermediate resistance to glycopeptides”, Circulation, 2003, 108: 767-71).

In a particular embodiment, the peptide of the invention is used to treat a microbial infection involving biofilm formation such as cystic fibrosis, endocarditis, cystitis, infections caused by indwelling medical devices, dental plaque formation or periodontitis.

In a preferred embodiment, the peptide of the invention is used to treat bacterial infections caused by multiple drug resistant bacteria. The bacterial infections to be treated include, for example, bacteremia, septicemia, skin and soft tissue infection, pneumonia, infection associated with an intravenous line or other catheter, canyl and/or device, superficial skin and/or mucous membrane infection. The bacterial infectious diseases include (but are not limited to) severe hospital-acquired infections, infections of the immunocompromised patients, infections of the organ transplant patients, infections at the intensive care units (ICU), severe infections of burn wounds, severe community-acquired infections, infections of cystic fibrosis patients.

The present invention also relates to a method for treating a microbial infection comprising administering a therapeutically effective dose of a peptide, a nucleic acid, a cassette or a vector according to the invention.

The term “therapeutically effective dose” as employed herein refers to the amount of peptide, nucleic acid, cassette or vector according to the invention required in order to observe an antimicrobial activity on the bacterium, virus, fungus or parasite responsible for the infection. The amount of peptide, nucleic acid, cassette or vector according to the invention to be administered and the duration of the treatment are determined by a person skilled in the art according to the physiological condition of the subject to be treated, the pathogenic agent and the antimicrobial activity of the peptide towards said pathogenic agent.

In still another aspect, the present invention relates to the use of a peptide according to the invention as disinfectant, preservative or pesticide.

The term “disinfectant” refers to an antimicrobial activity of the peptide on a surface (for example, walls, doors, medical equipment), a liquid (for example, water) or a gas (for example, an anaesthetic gas).

According to one embodiment, the peptide according to the invention is used for elimination of bacterial biofilms. According to a preferred embodiment, the peptide according to the invention is used in particular for disinfecting surgical or prosthetic equipment.

The present invention also relates to a medical device or implant comprising a body having at least one surface coated with or including an AMP according to the invention. The present invention also relates to a method for preparing a medical device or implant comprising applying a coating of peptide according to the invention, or placing in contact, with at least one surface of the device or implant.

This type of medical device or implant and the uses and methods of preparation thereof are described for example in patent application WO 2005/006938.

The surface coated with or including a peptide according to the invention may be composed of thermoplastic or polymeric materials such as polyethylene, Dacron, nylon, polyesters, polytetrafluoroethylene, polyurethane, latex, silicone elastomers and the like, or of metallic materials such as gold. In a particular embodiment, the peptide of the invention is covalently attached to a functionalized surface, preferably a metallic surface, via its N-terminal or C-terminal end. Optionally, the peptide may be attached to the surface through a spacer arm.

Preferably, the surface may be coated with a peptide at a density of 0.4 to 300 mg/cm².

Alternatively, the device or implant, in particular bone and joint prosthetic device, may be coated with a cement mixture comprising a peptide according to the invention.

The peptide may be combined with another active molecule, preferably an antibiotic.

The device or implant may be, for example, intravascular, peritoneal, pleural and urological catheters; heart valves; cardiac pacemakers; vascular shunts; coronary stunts; dental implants or orthopedic or intraocular prosthesis.

The present invention relates to a food composition comprising at least one peptide according to the invention.

Food products may be treated with a peptide according to the invention in order to eliminate or prevent the risk of infection by microorganisms and thereby improve their conservation. In this case the peptide is used as preservative.

The peptide according to the invention may be used as pesticide. In this case the peptide is used to prevent or treat infections of plants by phytopathogens.

The present invention also relates to an agrochemical composition comprising at least one peptide according to the invention.

The AMP according to the invention exhibits an antimicrobial activity and a similar cytolytic activity by comparison with temporin-SHf which are not considered as harmful with a LC50>200 μM. Preferably, the AMP according to the invention exhibits no or weak cytolytic activity. In particular, the peptide of the invention may have a LC50 of more than 30 μM for erythrocytes; preferably more than 40, 50, 100, 200, 500, 600, 800 μM. The LC50 value may be obtained for example on rat, dog, rabbit, pig, cat or human erythrocytes, preferably on rat or human erythrocytes, more preferably on human erythrocytes.

The term “lethal concentration, 50%” or “LC50” as employed herein refers to the concentration of substance required to kill half a population. LC50 is a quantitative indicator of the toxicity of a substance.

In addition to reduced cytotoxicity, the peptide of the invention has an antimicrobial activity that is preferably equal or superior to that of temporin-SHf against at least one bacterial, viral, fungal or parasitic strain.

Advantageously, the SHf analogs of the invention have an antimicrobial activity against Gram-negative and Gram-positive bacteria and are also non-cytotoxic. Their small sizes allow an easier synthesis.

The present invention will be better understood with the aid of the additional description which follows, which refers to non-limiting Example illustrating the synthesis of analogs and the antimicrobial tests.

### EXAMPLES

### Materials and Methods

### Peptide Synthesis

All SHf analogs were synthesized using solid-phase standard Fmoc chemistry protocols, as previously described (Raja et al., “Structure, antimicrobial activities and mode of interaction with membranes of novel phylloseptins from the painted-belly leaf frog, Phyllomedusa sauvagii”, PLoS One, 2013, 8:e70782) but with the following modifications. Synthesis was carried out on a CEM Liberty Blue automated microwave peptide synthesizer (CEM Corporation, Peptide Synthesis Platform, IBPS, Sorbonne University, Paris, France) using Protide Rink Amide LL resin (CEM Corporation, USA, 0.19 mmol/g substitution). Post-deprotection washing with N,N-dimethylformamide (DMF) was followed by coupling using a diisopropyl carbodiimide (DIC)/Oxyma activation method. The peptidyl resin was cleaved and deprotected by incubation (3 h at room temperature) with an acidic mixture containing 94% trifluoroacetic acid (TFA), 1% triisopropylsilane (TIS), 2.5% H₂O and 2.5% 1,2-ethanedithiol (EDT). Resin was removed by filtration and the peptide was precipitated in cold ether. The crude material was then subjected to semi-preparative RP-HPLC on a Phenomenex Luna® C18(2) semi-preparative column (10 μm, 250×10 mm) eluted at a flow rate of 5 mL/min by a 20-70% linear gradient of acetonitrile (0.07% TFA) in 0.1% TFA/water (1% acetonitrile/min). Peptide purity was assessed by analytical RP-HPLC, followed by MALDI-TOF analysis (Mass Spectrometry and Proteomics Platform, IBPS, Sorbonne University, Paris, France).

### Bacterial and Yeast Strains

The following strains were used:
Gram-negative bacteria: Escherichia coli ATCC 25922, Pseudomonas aeruginosa ATCC 27853, Acinetobacter baumannii ATCC 19606, Klebsiella pneumoniae ATCC 13883,
Gram-positive bacteria: Staphylococcus aureus ATCC 25923, multi-drug resistant Staphylococcus aureus ATCC BAA-44, Streptococcus pyogenes ATCC 19615, Listeria ivanovii Li4pVS2, Enterococcus faecalis ATCC 29212,

### Tests of Antibacterial Activity

For each strain, a standard inoculum of approximately 10⁶ bacteria/mL (exponential growth phase) was prepared. To this end, a colony isolated on LB agar previously inoculated with one of the strains was cultured in 4 ml of LB broth medium, except for S. pyogenes and L. ivanovii which were grown in BHI (Brain Heart Infusion) from a colony isolated on BHI agar. Liquid cultures were then incubated for 2 to 3 hrs at 37° C. with shaking for the bacteria to reach exponential growth phase. After centrifugation, most of the bacterial suspensions were diluted in Mueller-Hinton (MH) broth medium to an OD₆₃₀ₙₘ of 0.01, which corresponds to a concentration of approximately 10⁶ cfu/mL (cfu: colony forming unit). A different medium was used for E. faecalis (LB) and for S. pyogenes and L. ivanovii (BHI).

The minimum inhibitory concentration (MIC) of each peptide was determined by a test of growth inhibition in broth medium. MIC is defined as the lowest concentration of peptide able to inhibit the growth of the bacterial strain tested after 18-24 hrs of incubation at 37° C. The test was performed in a sterile 96-well microtiter plate. A series of increasing concentrations of peptide (2 to 400 μM) was first prepared in sterile MilliQ water. 50 μL of each peptide concentration were mixed into the well with 50 μL of bacterial suspension (10⁶ cfu/ml). The microtiter plate was then incubated for 18-24 hrs at 37° C. with shaking. Bacterial growth was determined by measuring OD at 630 nm (turbidity) on a plate reader. Tests were carried out in triplicate for each peptide concentration and at least three independent experiments were performed to determine the MIC value.

The growth inhibition negative control was obtained by replacing the solution containing the peptide with 50 μL of sterile MilliQ water. The positive control allowing the complete inhibition of bacterial growth was obtained by replacing the solution containing the peptide with 50 μL, of 0.7% formaldehyde.

### Cytotoxicity Assay

Hemolytic experiments were performed using human erythrocytes obtained from healthy adult donors (Etablissement Français du sang, Paris, France) according to a previously described protocol (Abbassi et al., “Isolation, characterization and molecular cloning of new temporins from the skin of the North African ranid Pelophylax saharica”, Peptides, 2008, 29: 1526-33).

Briefly, synthetic peptides (1-200 μM, final concentrations) were incubated (100 μL, final volume) with erythrocytes (2×10⁷ cells) in Dulbecco's phosphate-buffered saline (pH 7.4) for 1 h at 37° C. After centrifugation (12,000×g, 15 s), the absorbance of the supernatant was measured at 450 nm. The LC50 value, which is the average concentration of peptide producing 50% hemolysis, was determined from three independent experiments carried out in triplicate with positive control (100% hemolysis) corresponding to 0.1% triton (v/v).

### Results

Biological activities (antimicrobial activities and cytotoxicity) of SHf, known SHf analogs and of AMPs of the invention are provided in the Table 2.

TABLE 2

| | | MIC (μM) | | | | | | | | LC₅₀ (μM) |
|---|---|---|---|---|---|---|---|---|---|---|
| SEQ ID | | Gram-negative bacteria | | | | Gram-positive bacteria | | | | Erythrocytes |
| No | Sequence | EC | PA | AB | KP | SA | SP | LI | EF | Human |
| 2 | FFFLSRIF_{amide} | 50 | >100 | >100 | >100 | 25 | 25 | 50 | 100 | >200 |
| 11 | F(p-^{t}BuF)FLRRIF_{amide} | 6.25 | 12.5 | 6.25 | 100 | 3 | 3 | 6.25 | 25 | >200 |
| 19 | FF(hF)LRRIF_{amide} | 12.5 | 25 | 25 | 25/50 | 6.25 | 12.5 | 5/6.25 | 15/25 | 170 |
| 25 | F(p-^{t}BuF)(hF)LRKIF_{amide} | 6.25 | 6.25 | 3 | 6.25/12. | 3 | NE | NE | 6.25/12.5 | <100 |
| 29 | F(p-^{t}BuF)RFRRFF_{amide} | 6.25 | 3/6.25 | 12.5 | 25 | 3 | NE | NE | >25 | <100 |
| 30 | F(p-^{t}BuF)KFRRFF_{amide} | 6.25 | 3/6.25 | 12.5 | 25 | 3 | NE | NE | 25 | ~100 |
| 32 | F(p-^{t}BuF)KFKKFF_{amide} | 6.25 | 3 | 6.25 | 25 | 3/6.25 | NE | NE | 12.5 | ~100 |
| 33 | FK(p-^{t}BuF)LKKIF_{amide} | 6.25 | 3 | 25 | 25 | 6.25 | NE | NE | 12.5 | ~200 |
| 34 | FFFFSRFF_{amide} | >100 | >100 | >100 | >100 | 12.5 | 6.25 | 25 | 100 | >200 |
| 38 | FFFRRRFF_{amide} | 25 | 25 | 100 | >100 | 6.25/12.5 | 12.5 | 15/25 | >100 | >200 |
| 39 | FFFFRRFF_{amide} | 12.5 | 12.5 | 25 | 50 | 6.25 | 3 | 12.5 | 50 | >200 |
| 41 | FFRRFFFF_{amide} | 12.5 | 12.5 | 12.5 | 25 | 6.25 | 6.25 | 10/12.5 | 50 | >200 |
| 42 | FFFFKRFF_{amide} | 12.5/15 | 12.5 | 12.5/15 | 50 | 6.25 | 12.5 | 12.5/15 | >100 | >200 |
| 43 | FFFFRKFF_{amide} | 12.5/15 | 12.5 | 12.5 | 50 | 6/12.5 | 12.5 | 10/12.5 | 100 | >200 |
| 49 | FRFFRRFF_{amide} | 20/25 | 6.25 | 25/50 | 100 | 3/6.25 | 25 | 10/12.5 | >100 | >200 |
| 50 | FFRFRRFF_{amide} | 12.5/15 | 12.5 | 100 | 100 | 6.25 | 6.25 | 10/12.5 | 100 | >200 |
| 51 | F(hF)FFRRFF_{amide} | 12.5/20 | 12.5/15 | 12.5 | 25 | 6/6.25 | 12.5 | 12.5 | >100 | 159 |
| 53 | FF(hF)FRRFF_{amide} | 12.5 | 12.5 | 12.5 | 50 | 3 | 6.25 | 5/6.25 | 50 | 140 |
| 55 | FRFRFRFF_{amide} | 5/6.25 | 5/6.25 | 12.5/15 | 12.5 | 3 | 12.5 | 5/6.25 | >100 | >200 |
| 56 | FRFRFRFR_{amide} | 3 | 3/6 | 25 | 12.5 | 6/6.25 | 6.25 | 5/6.25 | 30 | >200 |
| 65 | (p-^{t}BuF)FKLRRIF_{amide} | 12.5 | NE | NE | NE | 1.56/3 | NE | NE | NE | >200 |
| 66 | (p-^{t}BuF)KFLRRIF_{amide} | 6.25 | 3 | 25 | 6.25 | 6.25 | NE | NE | 12.5 | 100-200 |
| 72 | RFFFRRFF_{amide} | 20/25 | 12.5 | 100 | 100 | 6.25 | 25 | 12.5/15 | >100 | >200 |

MIC: Minimum Inhibitory Concentration
LC50: Lytic Concentration 50%
EC: Escherichia coli; PA: Pseudomonas aeruginosa; AB: Acinetobacter baumannii; KP: Klebsiella pneumoniae; SA: Staphylococcus aureus; SP: Streptococcus pyogenes; LI: Listeria ivanovii; EF: Enterococcus faecalis.
NE: Not evaluated.

1. An antimicrobial peptide or a pharmaceutically acceptable salt thereof, wherein the antimicrobial peptide comprises the sequence:
(SEQ ID NO: 1)
X1-X2-X3-X4-X5-X6-X7-X8,
wherein:
X1 is an amino acid selected from the group consisting of: F, hF, 4a-F, (C₁-C₄ alkyl)F, W, R, and K,
X2 is an amino acid selected from the group consisting of: F, I, W, hF, K, (C₁-C₄ alkyl)F, L, and R,
X3 is an amino acid selected from the group consisting of: F, K, hF, R, (C₁-C₄ alkyl)F, and W,
X4 is an amino acid selected from the group consisting of: L, F, R, hF, and W,
X5 is an amino acid selected from the group consisting of: S, HmS, R, K, and hF,
X6 is an amino acid selected from the group consisting of: R and K,
X7 is an amino acid selected from the group consisting of: I, F, R, W, and hF, and
X8 is an amino acid selected from the group consisting of: F_{amide} and R_{amide}; and
wherein peptides of SEQ ID NOs: 2 to 13 are excluded.

2. The antimicrobial peptide according to claim 1, wherein:
X1 is an amino acid selected from the group consisting of: F, hF, p-^{t}BuF, 4a-F, W, R, and K,
X2 is an amino acid selected from the group consisting of: F, I, W, hF, K, p-^{t}BuF, L, and R,
X3 is an amino acid selected from the group consisting of: F, K, hF, R, p-^{t}BuF, and W,
X4 is an amino acid selected from the group consisting of: L, F, R, hF, and W,
X5 is an amino acid selected from the group consisting of: S, HmS, R, K, and hF,
X6 is an amino acid selected from the group consisting of: R, and K,
X7 is an amino acid selected from the group consisting of: I, F, R, W, and hF, and
X8 is an amino acid selected from the group consisting of: F_{amide} and R_{amide}.

3. The antimicrobial peptide according to claim 1, wherein said peptide has a net positive charge.

4. The antimicrobial peptide according to claim 3, wherein said net positive charge is at least +2, or is between +2 to +5.

5. The antimicrobial peptide according to claim 1, wherein the value of hydrophobicity of said peptide is comprised from 50 to 80%.

6. The antimicrobial peptide according to claim 1, wherein said peptide comprises at least three amino acids F.

7. The antimicrobial peptide according to claim 1, wherein said peptide is cyclic.

8. The antimicrobial peptide according to claim 1, wherein said peptide: comprises 3 F and 2 R or 3 K; or comprises 4 F and 1 or 2 or 4 R; or comprises 5 F and 2 or 3 R or 3 K; or comprises 6 F and 1 R or 2 R.

9. The antimicrobial peptide according to claim 8, wherein at least one amino acid F of said peptide is substituted by a hF and/or by a (C₁-C₄ alkyl)F.

10. The antimicrobial peptide according to claim 9, wherein (C₁-C₄ alkyl)F is p-^{t}BuF.

11. The antimicrobial peptide according to claim 1, wherein said peptide comprises an amino sequence selected from the group consisting of: SEQ ID NO: 14 to SEQ ID NO: 79.

12. The antimicrobial peptide according to claim 1, wherein said peptide comprises an amino sequence selected from the group consisting of: SEQ ID NOs: 19, 25, 29, 30, 32, 33, 34, 38, 39, 41, 42, 43, 49, 50, 51, 53, 55, 56, 65, 66, and 72; or wherein said peptide comprises an amino acid sequence of SEQ ID NO: 55 or SEQ ID NO: 56.

13. The antimicrobial peptide according to claim 1, wherein:
X1 is the amino acid F,
X2 is the amino acid p-^{t}BuF,
X3 is an amino acid selected from the group consisting of: K, hF and R,
X4 is an amino acid selected from the group consisting of: L and F,
X5 is the amino acid R,
X6 is an amino acid selected from the group consisting of: R and K,
X7 is an amino acid selected from the group consisting of: I and F, and
X8 is the amino acid F_{amide}.

14. A pharmaceutical composition comprising at least one antimicrobial peptide according to claim 1, and a pharmaceutically acceptable support and/or excipient.

15. The antimicrobial peptide according to claim 1, wherein the antimicrobial peptide is formulated as a pharmaceutical composition as a medicament.

16. The antimicrobial peptide according to claim 1, wherein the antimicrobial peptide is formulated as a pharmaceutical composition for preventing and/or treating an infection due to a bacterium, a virus, a fungus, or a parasitic.

17. The antimicrobial peptide according to claim 1, wherein said peptide is a disinfectant, preservative or pesticide.


SEQUENCE LISTING (79 sequences)

SEQ ID NO: 1 - protein, 8 aa, Artificial Sequence
  derived from Pelophylax saharicus
  VARIANT (1)..(1): replace by F or hF or 4a-F or (C1-C4 alkyl)F or W or R or K
  VARIANT (2)..(2): replace by F or I or W or  hF or K or (C1-C4 alkyl)F or L or R
  VARIANT (3)..(3): replace by F or  K or hF or  R or (C1-C4 alkyl)F or W
  VARIANT (4)..(4): replace by L or F or R or hF or W
  VARIANT (5)..(5): replace by S or HmS or R or K or hF
  VARIANT (6)..(6): replace by R or K
  VARIANT (7)..(7): replace by I or F or R or W or hF
  VARIANT (8)..(8): replace by Famide or Ramide
XXXXXXXX

SEQ ID NO: 2 - protein, 8 aa, Artificial Sequence
  derived from Pelophylax saharicus
  VARIANT (8)..(8): replace by Famide
FFFLSRIF

SEQ ID NO: 3 - protein, 8 aa, Artificial Sequence
  derived from Pelophylax saharicus
  VARIANT (8)..(8): replace by Famide
FFFLRRIF

SEQ ID NO: 4 - protein, 8 aa, Artificial Sequence
  derived from Pelophylax saharicus
FFFLRRIF

SEQ ID NO: 5 - protein, 8 aa, Artificial Sequence
  derived from Pelophylax saharicus
  VARIANT (8)..(8): replace by Famide
FSFLSRIF

SEQ ID NO: 6 - protein, 8 aa, Artificial Sequence
  derived from Pelophylax saharicus
  VARIANT (5)..(5): replace by HmS
  VARIANT (8)..(8): replace by Famide
FFFLSRIF

SEQ ID NO: 7 - protein, 8 aa, Artificial Sequence
  derived from Pelophylax saharicus
  VARIANT (3)..(3): replace by alphaMeF
  VARIANT (8)..(8): replace by Famide
FFFLSRIF

SEQ ID NO: 8 - protein, 8 aa, Artificial Sequence
  derived from Pelophylax saharicus
  VARIANT (1)..(1): replace by p-tBuF
  VARIANT (8)..(8): replace by Famide
FFFLSRIF

SEQ ID NO: 9 - protein, 8 aa, Artificial Sequence
  derived from Pelophylax saharicus
  VARIANT (1)..(1): replace by p-tBuF
  VARIANT (8)..(8): replace by Famide
FFFLRRIF

SEQ ID NO: 10 - protein, 8 aa, Artificial Sequence
  derived from Pelophylax saharicus
  VARIANT (2)..(2): replace by p-tBuF
  VARIANT (8)..(8): replace by Famide
FFFLSRIF

SEQ ID NO: 11 - protein, 8 aa, Artificial Sequence
  derived from Pelophylax saharicus
  VARIANT (2)..(2): replace by p-tBuF
  VARIANT (8)..(8): replace by Famide
FFFLRRIF

SEQ ID NO: 12 - protein, 8 aa, Artificial Sequence
  derived from Pelophylax saharicus
  VARIANT (3)..(3): replace by p-tBuF
  VARIANT (8)..(8): replace by Famide
FFFLSRIF

SEQ ID NO: 13 - protein, 8 aa, Artificial Sequence
  derived from Pelophylax saharicus
  VARIANT (3)..(3): replace by p-tBuF
  VARIANT (8)..(8): replace by Famide
FFFLRRIF

SEQ ID NO: 14 - protein, 8 aa, Artificial Sequence
  derived from Pelophylax saharicus
  VARIANT (8)..(8): replace by Famide
FFFLKKIF

SEQ ID NO: 15 - protein, 8 aa, Artificial Sequence
  derived from Pelophylax saharicus
  VARIANT (8)..(8): replace by Famide
FIFLRRIF

SEQ ID NO: 16 - protein, 8 aa, Artificial Sequence
  derived from Pelophylax saharicus
  VARIANT (8)..(8): replace by Famide
FWFLRRIF

SEQ ID NO: 17 - protein, 8 aa, Artificial Sequence
  derived from Pelophylax saharicus
  VARIANT (2)..(2): replace by hF
  VARIANT (8)..(8): replace by Famide
FFKLRRIF

SEQ ID NO: 18 - protein, 8 aa, Artificial Sequence
  derived from Pelophylax saharicus
  VARIANT (3)..(3): replace by hF
  VARIANT (8)..(8): replace by Famide
FFFLSRIF

SEQ ID NO: 19 - protein, 8 aa, Artificial Sequence
  derived from Pelophylax saharicus
  VARIANT (3)..(3): replace by hF
  VARIANT (8)..(8): replace by Famide
FFFLRRIF

SEQ ID NO: 20 - protein, 8 aa, Artificial Sequence
  derived from Pelophylax saharicus
  VARIANT (2)..(2): replace by hF
  VARIANT (8)..(8): replace by Famide
FFFLRRIF

SEQ ID NO: 21 - protein, 8 aa, Artificial Sequence
  derived from Pelophylax saharicus
  VARIANT (3)..(3): replace by hF
  VARIANT (8)..(8): replace by Famide
FFFLRKIF

SEQ ID NO: 22 - protein, 8 aa, Artificial Sequence
  derived from Pelophylax saharicus
FRFRFRFR

SEQ ID NO: 23 - protein, 8 aa, Artificial Sequence
  derived from Pelophylax saharicus
  VARIANT (3)..(3): replace by hF
  VARIANT (8)..(8): replace by Famide
FKFLKKIF

SEQ ID NO: 24 - protein, 8 aa, Artificial Sequence
  derived from Pelophylax saharicus
  VARIANT (2)..(2): replace by p-tBuF
  VARIANT (3)..(3): replace by hF
  VARIANT (8)..(8): replace by Famide
FFFLRRIF

SEQ ID NO: 25 - protein, 8 aa, Artificial Sequence
  derived from Pelophylax saharicus
  VARIANT (2)..(2): replace by p-tBuF
  VARIANT (3)..(3): replace by hF
  VARIANT (8)..(8): replace by Famide
FFFLRKIF

SEQ ID NO: 26 - protein, 8 aa, Artificial Sequence
  derived from Pelophylax saharicus
  VARIANT (2)..(2): replace by p-tBuF
  VARIANT (3)..(3): replace by hF
  VARIANT (8)..(8): replace by Famide
FFFLKKIF

SEQ ID NO: 27 - protein, 8 aa, Artificial Sequence
  derived from Pelophylax saharicus
  VARIANT (2)..(2): replace by p-tBuF
  VARIANT (8)..(8): replace by Famide
FFFFRRFF

SEQ ID NO: 28 - protein, 8 aa, Artificial Sequence
  derived from Pelophylax saharicus
  VARIANT (2)..(2): replace by p-tBuF
  VARIANT (8)..(8): replace by Famide
FFFFKRFF

SEQ ID NO: 29 - protein, 8 aa, Artificial Sequence
  derived from Pelophylax saharicus
  VARIANT (2)..(2): replace by p-tBuF
  VARIANT (8)..(8): replace by Famide
FFRFRRFF

SEQ ID NO: 30 - protein, 8 aa, Artificial Sequence
  derived from Pelophylax saharicus
  VARIANT (2)..(2): replace by p-tBuF
  VARIANT (8)..(8): replace by Famide
FFKFRRFF

SEQ ID NO: 31 - protein, 8 aa, Artificial Sequence
  derived from Pelophylax saharicus
  VARIANT (2)..(2): replace by p-tBuF
  VARIANT (8)..(8): replace by Famide
FFFFKKFF

SEQ ID NO: 32 - protein, 8 aa, Artificial Sequence
  derived from Pelophylax saharicus
  VARIANT (2)..(2): replace by p-tBuF
  VARIANT (8)..(8): replace by Famide
FFKFKKFF

SEQ ID NO: 33 - protein, 8 aa, Artificial Sequence
  derived from Pelophylax saharicus
  VARIANT (3)..(3): replace by p-tBuF
  VARIANT (8)..(8): replace by Famide
FKFLKKIF

SEQ ID NO: 34 - protein, 8 aa, Artificial Sequence
  derived from Pelophylax saharicus
  VARIANT (8)..(8): replace by Famide
FFFFSRFF

SEQ ID NO: 35 - protein, 8 aa, Artificial Sequence
  derived from Pelophylax saharicus
  VARIANT (8)..(8): replace by Famide
FFRFSRFF

SEQ ID NO: 36 - protein, 8 aa, Artificial Sequence
  derived from Pelophylax saharicus
  VARIANT (8)..(8): replace by Famide
FFKFSRFF

SEQ ID NO: 37 - protein, 8 aa, Artificial Sequence
  derived from Pelophylax saharicus
  VARIANT (2)..(2): replace by hF
  VARIANT (8)..(8): replace by Famide
FFFFSRFF

SEQ ID NO: 38 - protein, 8 aa, Artificial Sequence
  derived from Pelophylax saharicus
  VARIANT (8)..(8): replace by Famide
FFFRRRFF

SEQ ID NO: 39 - protein, 8 aa, Artificial Sequence
  derived from Pelophylax saharicus
  VARIANT (8)..(8): replace by Famide
FFFFRRFF

SEQ ID NO: 40 - protein, 8 aa, Artificial Sequence
  derived from Pelophylax saharicus
  VARIANT (5)..(6): replace by R in D configuration
  VARIANT (8)..(8): replace by Famide
FFFFRRFF

SEQ ID NO: 41 - protein, 8 aa, Artificial Sequence
  derived from Pelophylax saharicus
  VARIANT (8)..(8): replace by Famide
FFRRFFFF

SEQ ID NO: 42 - protein, 8 aa, Artificial Sequence
  derived from Pelophylax saharicus
  VARIANT (8)..(8): replace by Famide
FFFFKRFF

SEQ ID NO: 43 - protein, 8 aa, Artificial Sequence
  derived from Pelophylax saharicus
  VARIANT (8)..(8): replace by Famide
FFFFRKFF

SEQ ID NO: 44 - protein, 8 aa, Artificial Sequence
  derived from Pelophylax saharicus
  VARIANT (8)..(8): replace by Famide
FFFFKKFF

SEQ ID NO: 45 - protein, 8 aa, Artificial Sequence
  derived from Pelophylax saharicus
  VARIANT (5)..(6): replace by k in D configuration
  VARIANT (8)..(8): replace by Famide
FFFFKKFF

SEQ ID NO: 46 - protein, 8 aa, Artificial Sequence
  derived from Pelophylax saharicus
  VARIANT (8)..(8): replace by Famide
FWFFRRFF

SEQ ID NO: 47 - protein, 8 aa, Artificial Sequence
  derived from Pelophylax saharicus
  VARIANT (8)..(8): replace by Famide
FFWFRRFF

SEQ ID NO: 48 - protein, 8 aa, Artificial Sequence
  derived from Pelophylax saharicus
  VARIANT (8)..(8): replace by Famide
FLFFRRFF

SEQ ID NO: 49 - protein, 8 aa, Artificial Sequence
  derived from Pelophylax saharicus
  VARIANT (8)..(8): replace by Famide
FRFFRRFF

SEQ ID NO: 50 - protein, 8 aa, Artificial Sequence
  derived from Pelophylax saharicus
  VARIANT (8)..(8): replace by Famide
FFRFRRFF

SEQ ID NO: 51 - protein, 8 aa, Artificial Sequence
  derived from Pelophylax saharicus
  VARIANT (2)..(2): replace by hF
  VARIANT (8)..(8): replace by Famide
FFFFRRFF

SEQ ID NO: 52 - protein, 8 aa, Artificial Sequence
  derived from Pelophylax saharicus
  VARIANT (2)..(2): replace by hF
  VARIANT (8)..(8): replace by Famide
FFKFRRFF

SEQ ID NO: 53 - protein, 8 aa, Artificial Sequence
  derived from Pelophylax saharicus
  VARIANT (3)..(3): replace by hF
  VARIANT (8)..(8): replace by Famide
FFFFRRFF

SEQ ID NO: 54 - protein, 8 aa, Artificial Sequence
  derived from Pelophylax saharicus
  VARIANT (3)..(3): replace by hF
  VARIANT (8)..(8): replace by Famide
FFFFKRFF

SEQ ID NO: 55 - protein, 8 aa, Artificial Sequence
  derived from Pelophylax saharicus
  VARIANT (8)..(8): replace by Famide
FRFRFRFF

SEQ ID NO: 56 - protein, 8 aa, Artificial Sequence
  derived from Pelophylax saharicus
  VARIANT (8)..(8): replace by Ramide
FRFRFRFR

SEQ ID NO: 57 - protein, 8 aa, Artificial Sequence
  derived from Pelophylax saharicus
  VARIANT (4)..(4): replace by hF
  VARIANT (7)..(7): replace by hF
  VARIANT (8)..(8): replace by Famide
FRFFRRFF

SEQ ID NO: 58 - protein, 8 aa, Artificial Sequence
  derived from Pelophylax saharicus
  VARIANT (5)..(5): replace by hF
  VARIANT (7)..(7): replace by hF
  VARIANT (8)..(8): replace by Ramide
FRFRFRFR

SEQ ID NO: 59 - protein, 8 aa, Artificial Sequence
  derived from Pelophylax saharicus
  VARIANT (3)..(3): replace by hF
  VARIANT (8)..(8): replace by Famide
FRFFRRFF

SEQ ID NO: 60 - protein, 8 aa, Artificial Sequence
  derived from Pelophylax saharicus
  VARIANT (3)..(3): replace by hF
  VARIANT (8)..(8): replace by Famide
FFFFKKFF

SEQ ID NO: 61 - protein, 8 aa, Artificial Sequence
  derived from Pelophylax saharicus
  VARIANT (3)..(3): replace by hF
  VARIANT (8)..(8): replace by Famide
FFFLKKIF

SEQ ID NO: 62 - protein, 8 aa, Artificial Sequence
  derived from Pelophylax saharicus
  VARIANT (4)..(4): replace by hF
  VARIANT (7)..(7): replace by hF
  VARIANT (8)..(8): replace by Famide
FFFFRRFF

SEQ ID NO: 63 - protein, 8 aa, Artificial Sequence
  derived from Pelophylax saharicus
  VARIANT (3)..(3): replace by hF
  VARIANT (8)..(8): replace by Famide
FFFLRRRF

SEQ ID NO: 64 - protein, 8 aa, Artificial Sequence
  derived from Pelophylax saharicus
  VARIANT (8)..(8): replace by Famide
FFWLRRRF

SEQ ID NO: 65 - protein, 8 aa, Artificial Sequence
  derived from Pelophylax saharicus
  VARIANT (1)..(1): replace by p-tBuF
  VARIANT (8)..(8): replace by Famide
FFKLRRIF

SEQ ID NO: 66 - protein, 8 aa, Artificial Sequence
  derived from Pelophylax saharicus
  VARIANT (1)..(1): replace by p-tBuF
  VARIANT (8)..(8): replace by Famide
FKFLRRIF

SEQ ID NO: 67 - protein, 8 aa, Artificial Sequence
  derived from Pelophylax saharicus
  VARIANT (1)..(1): replace by 4aF
  VARIANT (8)..(8): replace by Famide
FFFFSRFF

SEQ ID NO: 68 - protein, 8 aa, Artificial Sequence
  derived from Pelophylax saharicus
  VARIANT (1)..(1): replace by 4aF
  VARIANT (2)..(2): replace by hF
  VARIANT (8)..(8): replace by Famide
FFFFSRFF

SEQ ID NO: 69 - protein, 8 aa, Artificial Sequence
  derived from Pelophylax saharicus
  VARIANT (1)..(1): replace by 4aF
  VARIANT (8)..(8): replace by Famide
FLFFSRFF

SEQ ID NO: 70 - protein, 8 aa, Artificial Sequence
  derived from Pelophylax saharicus
  VARIANT (1)..(1): replace by 4aF
  VARIANT (8)..(8): replace by Famide
FLFFRRFF

SEQ ID NO: 71 - protein, 8 aa, Artificial Sequence
  derived from Pelophylax saharicus
  VARIANT (8)..(8): replace by Famide
WFFFRRFF

SEQ ID NO: 72 - protein, 8 aa, Artificial Sequence
  derived from Pelophylax saharicus
  VARIANT (8)..(8): replace by Famide
RFFFRRFF

SEQ ID NO: 73 - protein, 8 aa, Artificial Sequence
  derived from Pelophylax saharicus
  VARIANT (4)..(4): replace by hF
  VARIANT (7)..(7): replace by hF
  VARIANT (8)..(8): replace by Famide
RFFFRRFF

SEQ ID NO: 74 - protein, 8 aa, Artificial Sequence
  derived from Pelophylax saharicus
  VARIANT (8)..(8): replace by Famide
RFFWRRWF

SEQ ID NO: 75 - protein, 8 aa, Artificial Sequence
  derived from Pelophylax saharicus
  VARIANT (3)..(3): replace by hF
  VARIANT (8)..(8): replace by Famide
RFFFRRFF

SEQ ID NO: 76 - protein, 8 aa, Artificial Sequence
  derived from Pelophylax saharicus
  VARIANT (3)..(3): replace by hF
  VARIANT (8)..(8): replace by Famide
KFFLKKIF

SEQ ID NO: 77 - protein, 8 aa, Artificial Sequence
  derived from Pelophylax saharicus
  VARIANT (1)..(1): replace by hF
  VARIANT (3)..(3): replace by hF
  VARIANT (5)..(5): replace by hF
  VARIANT (7)..(7): replace by hF
  VARIANT (8)..(8): replace by Ramide
FRFRFRFR

SEQ ID NO: 78 - protein, 8 aa, Artificial Sequence
  derived from Pelophylax saharicus
  VARIANT (1)..(1): replace by hF
  VARIANT (8)..(8): replace by Famide
FFFLRRIF

SEQ ID NO: 79 - protein, 8 aa, Artificial Sequence
  derived from Pelophylax saharicus
  VARIANT (1)..(1): replace by hF
  VARIANT (8)..(8): replace by Famide
FFKLRRIF